(19) United States
(12) Patent Application Publication
(10) Pub. No.: US 2016/0184810 A1
(43) Pub. Date: Jun. 30, 2016

(54) DEHYDRATION CATALYST AND METHOD OF MAKING AND USING THE SAME

(71) Applicant: White Dog Labs, Inc., New Castle, DE (US)

(72) Inventors: Michael E. WRIGHT, Ridgecrest, CA (US); Aharon M. EYAL, Jerusalem (IL)

(73) Assignee: White Dog Labs, Inc., New Castle, DE (US)

(21) Appl. No.: 14/969,838

(22) Filed: Dec. 15, 2015

Related U.S. Application Data
(60) Provisional application No. 62/097,519, filed on Dec. 29, 2014.

(51) Int. Cl. B01J 31/02; B01J 27/16; C07C 2/06; B01J 37/00; B01J 37/08; C07C 1/24; C08F 136/02; B01J 37/04

(52) U.S. Cl. CPC B01J 31/0274; C08F 136/02; B01J 27/16; B01J 37/04; B01J 37/009; B01J 37/08; C07C 1/24; C07C 2/06; C07C 2531/02

(57) ABSTRACT

A silanized surface, stable selective dehydration catalyst for dienes production, a method for producing a silanized surface, stable, selective dehydration catalyst, and a method for dehydrating at least one of an alkenol and a diol to a diene. The present invention also relates to the fermentation of sugars to afford bio-alkenols that are then separated and converted to a diene using a dehydration catalyst and a method for converting alkenols or diols to dienes with high selectivity and conversion. The dienes are useful in preparing synthetic rubber, plastics copolymers, and fuels.

### CROSS-REFERENCE TO RELATED APPLICATIONS

The present application is a non-provisional of U.S. Provisional Application No. 62/097,519 filed Dec. 29, 2015, the disclosure of which is expressly incorporated by reference herein in its entirety.

### FIELD OF THE INVENTION

The field of art to which the invention generally pertains is catalyst materials, including catalyst materials particularly useful in dehydration reactions.

### BACKGROUND

The energy and cost-efficient conversion of non-food biomass and/or waste gases to strategic chemical intermediates is one major step in securing a sustainable future for the world's population. Lignocellulosic materials contain a plentiful source of mixed sugars (C5 and C6) that can be obtained from non-food sources (e.g. corn stover and bagasse, waste products from food production) and thus can serve as one of many feedstocks for anaerobic fermentations by certain bacteria. Of particular value are the Clostridium species since they enjoy a robust and naturally occurring ability to use C5 and/or C6 sugars simultaneously. Furthermore, Clostridium is also well suited for the utilization of waste gases such as carbon monoxide and carbon dioxide as a carbon source for creating larger, more reduced hydrocarbons.

The acetone, butanol, and ethanol (ABE) biosynthetic pathways in Clostridium include well-established bio-catalytic steps where the key intermediates in the pathway are well characterized:

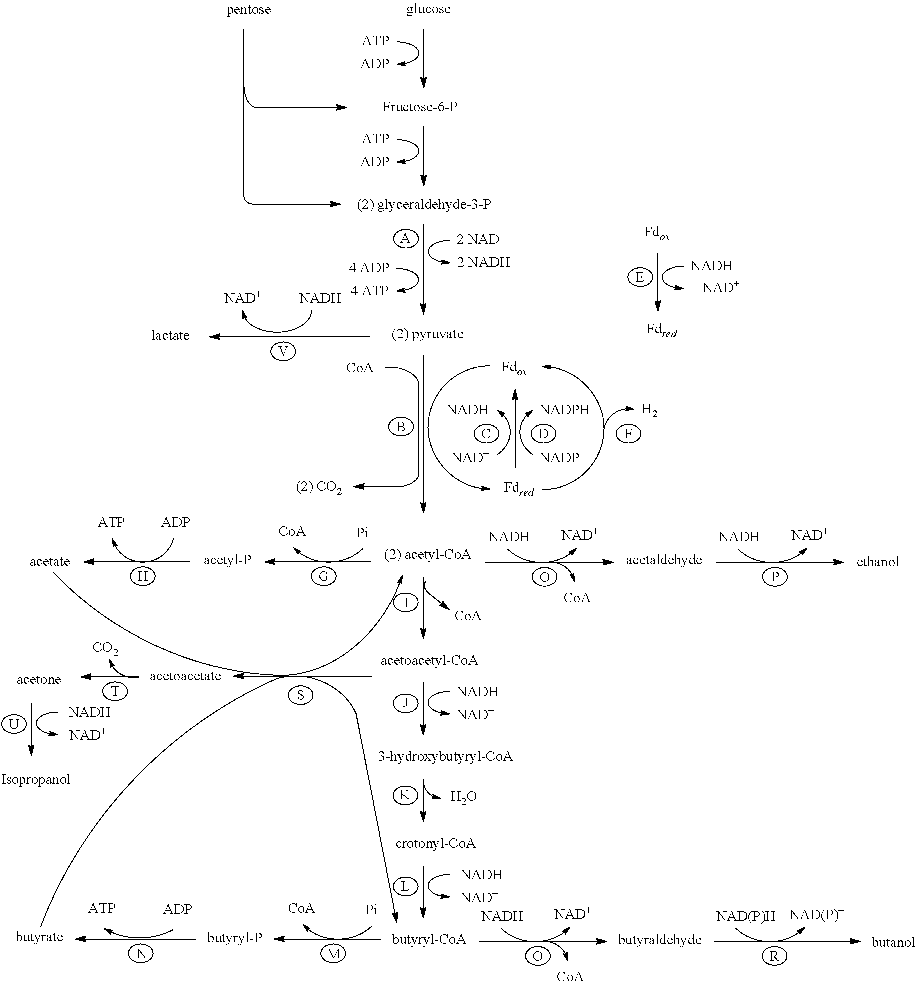

Well known to those skilled in the art of microbiology the Clostridium ABE process/pathway to bio-1-butanol has a key intermediate that is in fact crotonyl-CoA or sometimes noted as crotyl-CoA:

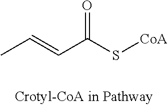

A number of renewable butadiene processes have been proposed, although none have been enabled. These include construction of metabolic pathways to produce butadiene or intermediates, thereof in a host organism. This requires introduction of multiple exogenous polynucleotides encoding multiple enzymes into a host organism. This in turn requires subsequent optimization of the expression of the multiple introduced genes and co-optimization of the kinetic profiles of the expressed genes to maximize flow of substrate to product. Therefore, there is a need in the art to develop strategies for renewable butadiene production where natural pathways in organisms are utilized to the fullest, co-product production is made advantageous, total solvent yields are increased, and efficient catalytic dehydration chemistry is employed to finally create the diene product.

α-Olefins are useful intermediates in preparing dienes, including 1,3-butadiene, diesel and jet/turbine fuels, lubricants, and polymers. 1-Butene is an especially useful precursor to 1,3-butadiene, which is used in preparing synthetic rubber and other advantageous polymeric elastomers. α-Olefins are also useful in preparing poly-α-olefins (PAOs) and copolymers with ethylene to form low-density plastics and with styrene to form elastomeric materials. Renewable α-olefin is useful in preparing the corresponding renewable products, including renewable fuels, polymers, elastomers, lubricants, PAOs, and other chemical intermediates. However, the conversion of 1-butene to 1,3-butadiene using the oxo-dehydrogenation process, although simplified over more traditional methods, still requires extensive purification and capital equipment, and also creates a significant chemical waste stream both in volume and complexity.

Currently, the production of ethylene from natural gas is rapidly replacing the steam cracking of naphthas and as a consequence, the four-carbon feedstocks (by-products like C4-raffinate, a mixture of butane, 1-butene, and 2-butenes) are rapidly shrinking in availability and creating uncertainty in cost. Thus, the traditional source of petroleum-based and non-renewable C4-feedstock, typically used for preparing butadiene is difficult to understand. It is this potential shortage and uncertainty of the petroleum feedstock that creates a need for a stable, renewable, and sustainable pathway to linear C4 platform molecules, for example 1,3-butadiene.

Obtaining α-olefins and dienes (e.g. 1,3-butadiene) from renewable and sustainable resources requires a significantly different approach than currently used in petroleum processes. Since alcohols can be produced at large scale by biological fermentation processes, they are an attractive feedstock for diene preparations, provided they can be chemically dehydrated in high conversion and selectivity to the desired diene product. This requires achieving high chemical selectivity in forming the alkene bonds and avoiding formation of carbon-oxygen double bonds (i.e. ketones or aldehydes) or the cleavage of carbon-carbon single bonds, or polymerization of the alkene/diene product. For example, it is known that the chemical dehydration of 3-buten-1-ol over solid phase catalysts leads to significant cleavage of the carbon-carbon bond and production of propene:

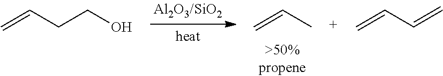

The conversion of oxygen-containing derivatives to butadiene has been studied for several decades and been the treatment of many literature reviews. For example, see the article written by Egloff & Hulk (UOP, Chem. Rev. 1945, 36(1), pp 63-141). Perhaps the most outstanding results to date in fact go back to the work reported by Reppe et al. in 1943 U.S. Pat. No. 2,310,809, the disclosure of which is herein incorporated by reference. This work indicated that 1,3-butanediol or 1, 4-butanediol could be doubly dehydrated over a combination of phosphoric acid, conjugate bases, and additional organic components to give butadiene in proposed yields of greater than 90%. However, the reported WHSVs are low relative to that acceptable for a viable industrial process and the lifetime of the catalyst is placed in weeks rather than many months. Furthermore, these results have not been reproduced in the last three decades where instrumentation, product detection, and structural verification have seen considerable advancements. Thus, there exists a need for a stable solid phase catalyst with sufficient activity, selectivity, and lifetime on stream that is in addition coupled with excellent documentation for the product stream, and in particular the diene product.

Recent work from the Sato laboratory at Chiba University in Japan has explored the dehydration of diols to alkenols, diols to butadiene, and the dehydration of alkenols to butadiene in significant detail. This work has focused on using a variety of different inorganic oxide catalysts, some modified with rare earth metal oxides. The work of Ichikawa et al. reported (J. Mol. Catal. A: Chem. 2006, 256, pp 106-112) on the dehydration of petroleum-derived 2-buten-1-ol (i.e. crotyl alcohol) is very telling. The best inorganic oxide catalyst was based on a SiO₂—Al₂O₃ composition

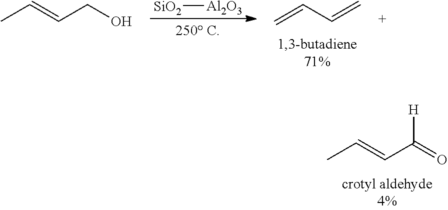

where the dehydration of crotyl alcohol afforded a 76% conversion with a 93% chemical selectivity for the diene, thus a 71% yield of butadiene:

Commercial production of bio-1-butanol has a rich history of successful large-scale production since the discovery by Louis Pasteur in the 1860s of bacteria that could ferment sugars to 1-butanol. Since Pasteur's initial discovery of the ABE process, many advances have been made in the fermentation process to optimize bio-1-butanol production and to reduce ethanol and acetone co-production. Most notable are the successful efforts using Clostridium bacteria in commercial plants developed by Weizmann at the turn of the 20^{th} century. With recent advances in fermentation and molecular biology strategies bio-1-butanol can be produced that is cost competitive to and even less than current petroleum-derived 1-butanol.

Since fermentations are carried out in aqueous medium, separation of the fermentation products of interest from the multi-component mixture (“fermentation broth”) of water, bacteria, media components, and other metabolic byproducts is energy- and time-intensive. In the case of bio-1-butanol, several methods have been reported for isolating the butanol component. Some methods use a sparging gas (e.g., carbon dioxide or steam) that carries the more volatile butanol/water azeotrope away from the fermentation broth while others involve the use of membrane technologies (i.e. pervaporization). In the latter, a selective membrane material is used that permits bio-1-butanol to pass through while leaving the bacteria, sugars, proteins, and a large proportion of the water behind. Regardless of the method, it is evident to those skilled in the art that removing water from the alcohol product is difficult (i.e. of high energy and capital cost). The available methods are numerous and vary drastically in capabilities. Clearly, water and impurities are a direct and unavoidable consequence of fermentation processes that produce alcohols. Ruwet et al. (Bull. Soc. Chim. 1987, 96, 281-292) discuss the problems in using a wet ABE 1-butanol feed in a dehydration reaction to afford a mixture of olefins. More recently, D'Amore et al. (US2008/0015395 A1) reported extreme difficulty in dehydrating aqueous solutions of 1-butanol using a variety of acid catalysts to afford a mixture of olefins and other oxygenated products (e.g., ethers), including high amounts of unreacted 1-butanol. Improved methods are needed to chemically dehydrate “wet” alkenols and diols to bio-dienes since the former are clearly the most accessible form of feedstock.

Internal alkenes are far less effective in Ziegler-Natta polymerization chemistry and are not of direct use in preparing plastics (e.g. low density polyethylene copolymers) and for making lubricants (e.g. poly alpha olefins, PAOs). Interestingly, butadiene can be an effective route to bio-1-butene by selective hydrogenation:

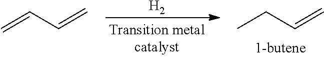

As a feedstock, it is highly desirable to have a selective process that affords rapid and high conversion to a terminal-olefin (e.g., bio-1-butene) and is accompanied by no carbon skeletal rearrangement (e.g., to isobutylene). Furthermore, bio-dienes such as bio-1,3-butadiene can be oligomerized/polymerized using a variety of methods known to those experienced in the art of organic synthesis to create rubbers, plastics, and fuels. Some products prepared from butadiene are hydroxyl end-capped oligomers which are commercial products used to make solid rocket motor binder systems.

1,3-butadiene is a useful intermediate for preparing several 6-carbon chemical intermediates, or monomers, that are useful in preparing polymers, for example nylon 6,6:

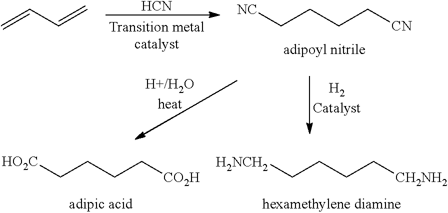

### BRIEF SUMMARY

A silanized surface, stable selective dehydration catalyst for dienes production is described including an inorganic support, at least one Bronsted acid of pKa less than 2.5, and at least one silicon compound.

Additional embodiments include, the catalyst described above further including at least one promoter and at least one conjugate base; the catalyst described above where the inorganic support is alumina, gamma alumina, silica or zinc aluminate; the catalyst described above where the inorganic support is gamma-alumina extrudate; the catalyst described above where the pKa of the Bronsted acid is in the range between about −7 and about 2.5; the catalyst described above where the Bronsted acid is phosphoric acid, sulfuric acid or combinations thereof; the catalyst described above where the promoter is at least one Group 1 metal, Group 2 metal or combination thereof; the catalyst described above where the conjugated base is at least one of HSO₄, SO₄, trimethylsilyl acetate, H₂PO₄⁻¹, HPO₄⁻², PO₄⁻³ or combinations thereof; the catalyst described above where the conjugate base is at least one of H₂PO₄⁻¹, HPO₄⁻², or combinations thereof; the catalyst described above where the silicon concentration is in the range of between about 0.01 wt % and about 2 wt % of said catalyst; and the catalyst described above where the promoter concentration is in the range of between about 0.2 wt % and about 5 wt % of said catalyst.

A method for producing a silanized surface, stable, selective dehydration catalyst, is also described including providing an inorganic support; mixing the inorganic support with a reagent solution including a solvent, a Bronstad acid, and an organosilane compound, where a support-solution mixture is formed; removing at least a portion of the solvent from the mixture to form a solvent-depleted mixture; and calcining the solvent-depleted mixture.

Additional embodiments include: the method described above where the organosilane compound is at least one of trimethylsilayl acetate, bis(trimethylsilyl) ether, diphenyldiethoxysilane, tri(trimethylsilyl)phosphate, or diphenyldiethoxysilane; the method described above where the solvent is ethanol, methanol, ethylene glycol, propanediol, propanol, iso-butanol, water or mixtures thereof; the method described above where the reagent solution further includes a promoter compound; the method described above where the calcining includes maintaining the solvent depleted mixture at a temperature in the range of between about 100° C. and about 500° C.; the method described above where the maintaining is for a time of between about 1 hour and about 10 hours; the method described above where the silicon content of the organosilane compound is in the range of between about 0.01 wt % and about 2 wt % of the support; and the method described above where the promoter content of the promoter compound is in the range of between about 0.2 wt % and about 5 wt % of the support.

A method for dehydrating an alkenol and/or a diol to a diene is also described, including providing a catalyst including an inorganic support, at least one Bronsted acid of pKa less than 2.5, and at least one silicon compound; providing a reactant mixture including at least one alkenol and/or diol; and contacting the reactant mixture with the catalyst at a WHSV of at least about 0.3 hr⁻¹ to selectively form a product composition comprising at least one product diene, where one-pass conversion yield is greater than about 80 mol %.

Additional embodiments include; the method described above where the WHSV is between about 0.3 hr⁻¹ and about 10 hr⁻¹; the method described above where the reactant mixture further includes water at a concentration between about 10 wt % and about 93 wt %; the method described above where the selectivity of the diene production is at least about 90%; the method described above where the silicon concentration in the provided catalyst is in the range between about 0.01 wt % and about 2 wt % of said catalyst; the method described above where the provided catalyst further includes at least one promoter and at least one conjugate base; the method described above where the promoter concentration in the provided catalyst is in the range of between about 0.2 wt % and about 5 wt % of the catalyst; the method described above where the alkenol includes crotyl alcohol; the method described above where the alkenol is a C₄ to C₆ alkenol; the method described above where the product diene includes 1,3-butadiene; the method described above where the contacting is conducted at a temperature in the range of between about 150° C. and about 350° C.; the method described above further including separating the diene from the product composition; the method described above where the separating includes distillation and/or solvent extraction; the method described above further including providing at least one purge gas; the method described above where the purge gas includes at least one of nitrogen, hydrocarbons, helium, argon and mixtures thereof; and the method described above including reacting the diene with at least one additional chemical to form a diene product.

A commercial product including diene product made according to the above method is also described. Additional embodiments include a commercial product including synthetic rubber, plastics, copolymers and fuels.

### BRIEF DESCRIPTION OF THE DRAWINGS

FIG. 1 illustrates a high level overall process for converting biomass to dienes

FIG. 2 illustrates a high-level process flow diagram showing the preparation of an invention dehydration catalyst.

FIG. 3 illustrates a high-level process flow diagram showing the preparation of a shaped invention dehydration catalyst.

FIG. 4 illustrates a high-level process flow diagram showing the dehydration of an alkenol or diol using the dehydration catalyst in a single reactor system to afford a diene.

FIG. 5 illustrates a high level process flow diagram showing the dehydration of an alkenol or diol using the dehydration catalyst in a two reactor system (i.e. adiabatic) to afford a diene.

FIG. 6 illustrates the acid catalyzed isomerization of crotyl alcohol to 2-buten-1-ol and subsequent dehydration of the later to 1,3-butadiene.

FIG. 7 illustrates a high level process diagram for the conversion of a four carbon alkenol or diol to six carbon monomers.

FIG. 8 shows proton NMR spectrum of the butadiene product made by an invention catalyst.

It is to be understood that the foregoing general description and the following detailed description are exemplary and explanatory only and are not to be viewed as being restrictive of the invention, as claimed. Further advantages of this invention will be apparent after a review of the following detailed description of the disclosed embodiments, which are illustrated schematically in the accompanying drawings and in the appended claims.

### DETAILED DESCRIPTION

The invention generally relates to net conversion of sugars and/or carbon containing gases to dienes, especially butadiene, by the efficient catalyzed thermal dehydration of alkenols and/or diols that can be prepared by fermentation, other biological processes, or chemical pathways. The dienes, including butadiene, are useful in the preparation of synthetic rubbers, plastics, fuels, and additional chemical intermediates incorporated into sustainable commercial products.

A strategy is described herein whereby a microorganism which can produce n-butanol is converted using one or more methods into a microorganism that produces crotyl alcohol. With this microorganism, crotyl alcohol can be produced by fermentation using existing fermenter designs and solvent extraction systems known to those skilled in the art for the isolation of butanol. Said crotyl alcohol can rapidly and inexpensively be converted to butadiene using a catalyst, also disclosed herein, thereby minimizing capital expenditure (CAPEX), reducing operating expenses (OPEX), and further enhancing the life cycle analysis (LCA) benefit to the process of converting biomass to bio-butadiene.

In some embodiments, the invention catalyst compositions and processes are provided to convert diols (e.g., bio-diols) to the corresponding dienes (e.g., bio-dienes). In other embodiments, alkenols (e.g., bio-alkenols) are converted to the corresponding dienes (e.g., bio-dienes). In additional embodiments, alkenols (e.g., bio-alkenols) are converted to a corresponding mixture of diene products (i.e. both terminal and internal alkenes). In one embodiment, 4-carbon alkenols (e.g., bio-alkenols) are converted to 1,3-butadiene (e.g., bio-1,3-butadiene). In specific embodiments, crotyl alcohol is converted to 1,3-butadiene. In some embodiments the feedstock (alcohol (e.g., bio-alcohol), alkenol (e.g., bio-alkenol), 4-carbon alkenol (e.g., bio-alkenol), crotyl alcohol, or diol) contains from about 0.1 wt-% to about 90 wt-% water and, using systems and processes disclosed herein, delivers a product stream containing the corresponding diene (e.g., bio-diene) and water. In some embodiments, the diene is produced with about 92% to about 99% chemical selectivity. In some embodiments, a single pass over the catalyst system affords a chemical conversion of greater than about 94%, or about 97%, or about 99.5%. In some embodiments, the diene (e.g., bio-diene) is produced employing WHSV values greater than 0.4 hr⁻¹, e.g., greater than 1 and less than 15 hr⁻¹, or about 0.4 hr⁻¹ to about 8 hr⁻¹. In some embodiments of the methods disclosed herein, at least one solid phase catalyst is maintained at a temperature of about 200° C. to about 440° C., or about 200° C. to about 320° C., with feed solution containing alkenol (e.g., bioalkenol) or diol (e.g., bio-diol) and water at a pressure of about 2 psig to about 2000 psig to produce a diene (e.g., bio-diene) in high chemical selectivity at very high alcohol conversion. In one embodiment, crotyl alcohol that is obtained from a fermentation broth (e.g., water saturated) is dehydrated to bio-1,3-butadiene in a chemical yield of greater than about 80%, or greater than 88%, or greater than 94% yield of butadiene in a single pass over the invention catalyst.

In one aspect, a method is provided for dehydrating an alkenol or a diol to a diene, including: contacting and heating the alkenol or diol with at least one catalyst that includes: i) an inorganic support, ii) an acid, and iii) a partially silanized surface at a WHSV at least about 0.4 hr⁻¹, thereby producing a diene product that includes one or more diene(s) at a one-pass chemical yield greater than about 75 mol %. In some embodiments, the WHSV is about 0.5 hr⁻¹ to about 8 hr⁻¹. In some embodiments, the alkenol or diol is in a mixture with about 10 ppm to about 90 wt % water, or about 10 wt % to about 60 wt % water. In some embodiments, the chemical selectivity of diene production is at least about 92%, for example, greater than 94%, or about 92% to about 98.5%. In some embodiments, the inorganic support includes γ-alumina, for example, in the form of a rod-like extrudate. In some embodiments, the catalyst is modified with at least one organosilane contained in solution. In one embodiment, the silanization solution does not contain a metal promoter; hence, the invention dehydration catalyst does not require a metal promoter to be useful in the fast and effective dehydration of alkenols to dienes.

In one aspect, a method is provided for dehydrating an alkenol or a diol to a diene, including: contacting and heating the alkenol or diol with at least one catalyst that includes: i) an inorganic support, and ii) an acid at a WHSV at least about 0.4 hr⁻¹, thereby producing a diene product that includes one or more diene(s) at a one-pass chemical yield greater than about 75 mol-%. In some embodiments, the WHSV is about 0.5 hr⁻¹ to about 8 hr⁻¹. In some embodiments, the alkenol or diol is in a mixture with about 100 ppm to about 90 wt % water, or about 5 wt % to about 60 wt % water. In some embodiments, the chemical selectivity of diene production is at least about 92%, for example, greater than 94%, or about 92% to about 98.5%. In some embodiments, the inorganic support includes γ-alumina, for example, in the form of an extrudate.

In another aspect, a method is provided for dehydrating an alkenol or a diol to a diene, including: contacting and heating the alkenol or diol with at least one catalyst that includes a silica containing inorganic support, one or more acids, and a silanzing agent thereby producing the corresponding diene product with greater than about 92 mol % chemical selectivity and greater than about 92 mol-% conversion in a single pass over the catalyst. Chemical yield of diene is the product of chemical selectivity in % times the mol-% conversion of the feed diol or alkenol. Thus, 0.92×0.92 would afford a 85% chemical yield of diene.

In some embodiments, catalyst compositions disclosed herein contain an inorganic support modified by a Mg⁺² compound is used. In one embodiment, modification may be accomplished via treatment of the support with a mixture of magnesium compounds, for example magnesium bis(hydrogensulfate), magnesium acetate, and/or magensium sulfate, dissolved in a solvent. In some embodiments Mg⁺² is the modifier and the Mg⁺² compound is magnesium bis(hydrogensulfate) [Mg(HSO₄)₂]. The solvent may be water, or an organic solvent, or a mixture of the two. In some embodiments, a solvent is used that is capable of completely dissolving the Mg⁺² compounds used for preparation of the catalyst. In some embodiments, an “Incipient Wetness Impregnation” (IWP) process is used for modifying the catalyst surface. In one embodiment, the catalyst composition includes Mg⁺² (as wt-% of the modified inorganic support) at about 0.2 to about 5.0 wt-%. In one embodiment, γ-alumina is used as the inorganic support. In some embodiments, γ-alumina can be modified with binder, physically formed (e.g., extrudate) to a preselected shape, calcined, and then modified with a mixture of Mg⁺² compounds, at least one acid, and at least one organosilane. In some embodiments, the shaped gamma-alumina is modified by treatment with at least one acid. In one embodiment, the shaped gamma-alumina is modified by treatment with at least one acid and at least one organosilane using at least one IWP solution.

In one aspect, a method of making a catalyst composition described herein is provided. The method includes modifying an inorganic support with a single solution containing: a) at least one Group 2 element, b) at least one buffer mixture of anions, c) and at least one organosilane, and to afford a fully functional invention catalyst after a final heat treatment at a temperature of about 100 to about 400° C. to remove at least some of the solvents.

In one embodiment, a promoter modified and calcined support may optionally be further modified by treatment with at least one acid to create a second catalyst that are especially useful for the dehydration of alkenols (e.g., bioalkenols) mixed with at least some a high steam. The steam can be introduced from 100 ppm to 90% by weight of the diol or alkenol. In some embodiments organosilane-modified catalysts afford high chemical selectivity for diene (e.g., bio-diene) products and can perform for extended periods of time on stream with little to no loss of product chemical selectivity and catalytic activity over a period of months or years.

In some embodiments, methods disclosed herein for producing terminal olefins include using [promoter-organosilane-acid]-modified alumina catalysts, as described herein, for example, in at least one isothermal continuous flow reactor. In one embodiment, a series of one or more adiabatic reactors are used to dehydrate an alkenol or diol to at least one diene product. In some embodiments, some of the heat necessary for the chemical dehydration reaction is carried into the reactor in the form of a gaseous diluent, such as steam or a mixture of at least one hydrocarbon or at least one inert gas. In the case of water addition, this can generate a very water rich gas phase in the reactor making some embodiments of the methods disclosed herein beneficial for commercial applications.

In some embodiments, methods disclosed herein for producing terminal olefins include using (organosilane and acid)-modified alumina catalysts, as described herein, for example, in at least one isothermal continuous flow reactor. In one embodiment, a series of one or more adiabatic reactors are used to dehydrate an alkenol or diol to at least one diene product. In some embodiments, some of the heat necessary for the chemical dehydration reaction is carried into the reactor in the form of a gaseous diluent, such as steam or a mixture of at least one hydrocarbon or at least one inert gas. In the case of water addition, this can generate a very water rich gas phase in the reactor making some embodiments of the methods disclosed herein beneficial for commercial applications.

In some embodiments, methods disclosed herein for producing terminal olefins include using an acid-modified alumina catalysts, as described herein, for example, in at least one isothermal continuous flow reactor. In one embodiment, a series of one or more adiabatic reactors are used to dehydrate an alkenol or diol to at least one diene product. In some embodiments, some of the heat necessary for the chemical dehydration reaction is carried into the reactor in the form of a gaseous diluent, such as steam or a mixture of at least one hydrocarbon or at least one inert gas.

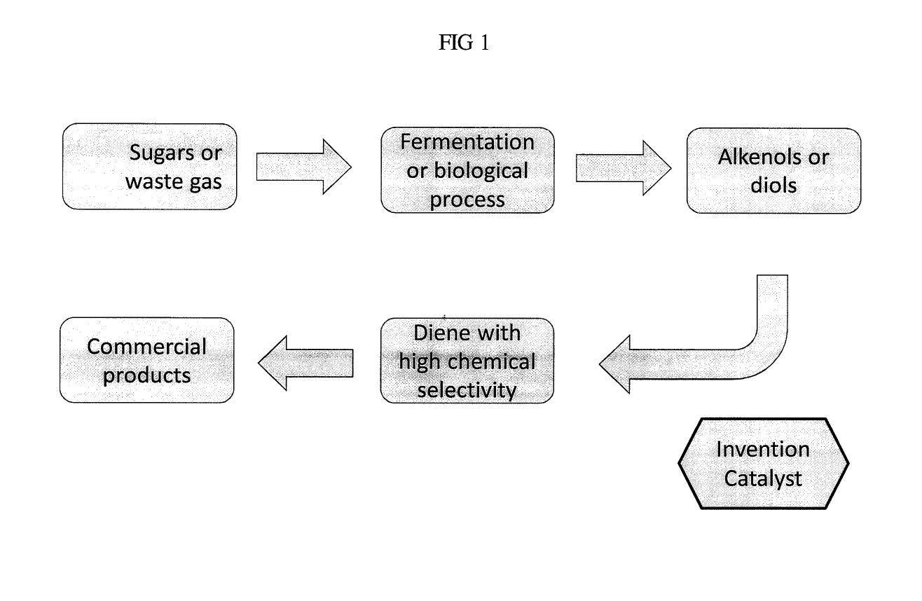

Some embodiments of the methods and systems described herein relate to fuels (e.g., alternative and/or renewable fuels) and the production of dienes [e.g., bio-dienes (e.g., biodiesels)] that can be utilized to prepare a drop-in and full performance diesel fuel (e.g., diesel biofuel), jet (e.g., biojet) fuel, lubricant (e.g., biolubricant), or other chemical products and/or intermediates useful in the production of commercial products. Embodiments also include such processes starting from cellulosic and/or hemicellulosic biomass material, as are shown schematically in FIG. 1. In one embodiment the process can start from dextrose or similar sugars that are formed as a by-product from corn processing. Bioalkenols or bio-diols can also be generated feeding a carbon-source of carbon dioxide and/or carbon monoxide to at least one biological organism. Typically the biological organism is suspended in water or it may be attached to at least one support system and then placed in contact with water and a carbon-source (e.g. dextrose, glucose, molasses, or carbon monoxide).

In one aspect, methods are provided for dehydrating an alkenol (e.g., bio-alkenol) to a diene (e.g., bio-diene). In some embodiments, the method includes contacting and heating an alkenol (e.g., bio-alkenol) with at least one catalyst that includes a calcium-modified inorganic support at a WHSV of about 0.4 hr⁻¹ to about 8 hr⁻¹, wherein the alkenol (e.g., bioalkenol) is in a mixture with about 1 ppm to about 90 wt % water, thereby producing the corresponding diene (e.g., bio-diene) with greater than about 90% selectivity and greater than 98% conversion.

In some embodiments of the methods disclosed herein, the diol (e.g., bio-diol) is a C₄ to C₁₂ bio-diol. In one embodiment, the alcohol (e.g., bio-alcohol) is butanediol (e.g., 1,3-butanediol). The diene (e.g., bio-diene) produced by some embodiments utilizes at least one invention catalyst disclosed herein is 1,3-butadiene. In some embodiments of the methods disclosed herein, the alcohol (e.g., bio-alcohol) is a diol (e.g., bio-diol) for example 1,3-butanediol.

In some embodiments of the methods disclosed herein, the alcohol (e.g., bio-alcohol) is an alkenol (e.g., bio-alkenol) such as 2-butene-1-ol (e.g., bio-2-buten-1-ol). For example, in some embodiments, the alkenol (e.g., bio-alkenol) or diol (e.g., bio-diol) may be a C₄ to C₁₈ alcohol (e.g. bio-1,3-butanediol or bio-1,12-decanediol). The diene produced in some embodiments of the methods disclosed herein may be 1,3-butadiene (e.g., bio-1,3-butadiene).

In some embodiments of the methods disclosed herein, the 1-alkenol is an alcohol of general formula R¹R²CH═CHCH₂OH, where R¹ and R² are independently H or a C₁ to C₂₀ alkyl radical, or contain one or more aromatic rings (e.g. R¹=phenyl and R²=Me) containing exclusively carbon or a mixture of carbon and selected heteroatoms (e.g. sulfur), or a nitrile, or an alkene, or a carboxylic acid, or a ketone group, or a combination thereof.

In some embodiments, the selectivity of diene (e.g., bio-diene) production is greater than about 95%. In some embodiments, the selectivity of diene (e.g., bio-diene) production is about 90% to about 99%. In some embodiments, the diene (e.g., bio-diene) product includes less than about 5% unreacted diol or alkenol.

In certain embodiments, water is present in the feed mixture to the catalyst at about 5 to about 70 wt-%, or about 10 to 20 wt-%, or about 17 wt-%.

The carbon-13 (C-13) content in the bio-1,3-butadiene prepared according to this invention is unique and easily measured and verified by techniques known to those skilled in the art of isotopic GC-mass spectrometer determination. The latter methods are very different from C-14 determination experimental determinations. The C-13 isotopic determinations can be performed in a routine manner and with great cost effectiveness. Furthermore, the C-13 isotopic compositions of said invention bio-dienes are unique to the source of feedstock. Each biomass (i.e. sugar) source will provide a distinct and measurable C-13/C-12 ratio in the bio-diene product made according to this invention.

In some embodiments, the inorganic support includes γ-alumina, or silica, or titanium oxide, or zinc aluminate, or combinations thereof. The surface area of the inorganic support may be in the range of about 40 to about 500 m²/g before surface modification. After addition of at least one acid, and/or at least one organosilane, and/or at least one promoter the range of surface area is about 30 to about 550 m²/g.

In some embodiments, the catalyst is modified by treatment with at least one organosilane that does not contain a promoter (e.g. Na), for example, an alkoxysilane (e.g. Me₃SiOMe). In one embodiment, the organosilane is trimethylsilylacetate. In another embodiment, the organosilane is bis(trimethylsilyl) ether. In some embodiments the organosilane has at least one alkyl chain or aromatic group attached to the silicon atom that contains at least one functional group that leads to increased water solubility, for example hydroxyl (i.e. —OH).

In some embodiments, the catalyst is modified by treatment with at least one organosilane and promoter dissolved in an alcohol solvent. In one embodiment, the organosilane is diphenyldiethoxysilane and alcohol solvent is ethanol. In another embodiment, the organosilane is diphenyldiethoxysilane and alcohol solvent is methanol containing 5 wt-% water. The alcohol solvent can contain water from about 10 ppm up to about 95 wt-%. For selected organosilane modifiers that possess adequate water solubility (i.e. greater than 0.5 wt-%), water alone can be used to deliver the organosilane and acid to the catalyst surface.

In some embodiments of the methods, at least one purge gas is provided, e.g., nitrogen and/or argon. In other embodiments the inert gas/purge gas (e.g. nitrogen) can have at least one hydrocarbon (e.g. butane, propane, hexane, etc.) added from 1 to 99 wt-% as a function of the alkenol or diol feed to the dehydration reactor.

In some embodiments, the reaction mixture is heated to a temperature of about 200° C. to about 440° C. In some embodiments, the temperature is about 200° C. to about 360° C. In some embodiments, the temperature is about 250° C. to about 320° C.

In some embodiments, the method further includes separating water from 1,3-butadiene product. In some embodiments, the method includes contacting the 1,3-butadiene product from which water has been removed with a second catalyst (hydrogenation type, e.g. palladium on carbon) and hydrogen gas to form 1-butene (e.g., bio-1-butene) in greater than 80% yield. Other more selective hydrogenation catalysts may also be used. The 1-butene (e.g., bio-1-butene) is oligomerized with a third catalyst. In some embodiments, the third catalyst may be a Ziegler-Natta catalyst, a mesoporous oligomerization catalyst, or another catalyst that is capable of producing oligomers from dienes (e.g., bio-dienes). In various embodiments, the oligomers may be used to produce a diesel fuel (e.g., with a flashpoint of about 38 to about 100° C., a Cetane rating of about 45 to about 60, and aromatic content of less than about 0.5 wt-%), a jet fuel (e.g., with a flashpoint of about 38 to about 100° C., a cold flow viscosity of less than about 8.0 cSt at −20° C., and aromatic content of less than about 0.5 wt-%), or a lubricant (e.g., with a viscosity of about 1 to about 10,000 cSt at 25° C.).

In some embodiments, the method includes contacting and heating an alcohol (e.g., bioalcohol) or diol (e.g., bio-diol) with at least one catalyst that includes an inorganic support that is modified by a promoter, an acid component, and at least one organosilane, thereby producing the corresponding diene with greater than about 90% chemical selectivity. For example, the Group 2 element (i.e. promoter) may include at least one of Mg, Ca, Ba, and Sr. Optionally, the alkenol (e.g., bioalkenol) or diol (e.g., bio-diol) is in a mixture with about 1 ppm to about 90 wt % water. Optionally, the catalyst is contacted with the diol (e.g., bio-diol) at a WHSV of greater than 0.4 (e.g., about 0.4 hr⁻¹ to about 12 hr⁻¹). In some embodiments Group 1 metals can be used in place of Group 2 elements and provide similar performance and time-on-stream. For example, this includes Li, Na, and K and mixtures of Group 1 and Group 2 metals as one embodiment.

In some embodiments, dehydration of crotyl alcohol (a bio-alkenol) using the catalyst compositions and methods described herein provides 1,3-butadiene (e.g., bio-1,3-butadiene) in greater than 90% chemical yield, and, in some embodiments, 90-99% chemical selectivity for 1,3-butadiene or 95-99% selectivity. In some embodiments, the catalyst can be used continuously for at least 1 to 3 months, or about 6 months and in some embodiments, up to about 12 months, or up to about 18 months, or periods of greater than 20 months while producing 1,3-butadiene without significant loss in chemical selectivity for the terminal diene.

In another aspect, the charge of the Group 2 element is balanced with at least one counter anion (i.e. X−) that can also deliver the requiste acid component. The pKa for at least one respective conjugate acid (i.e. XH) is the range of about −7 to about 2.5 or about 1.5 to about 2.0. The composition of the Group 2 element may use a mixture of counter anions that have one or more pKa values for each respective conjugate acid. In one embodiment then the Group 2 element is matched with counter anions that effectively form a silanized surface on the inorganic support. For example, a mixture containing both Ca(HSO₄)₂, Ca(SO₄), and trimethylsilanol can be used to deliver an acid, a promoter, and silanizing agent to the catalyst surface as a single IWP solution. Another embodiment can use a mixture of phosphates (H₃PO₄, H₂PO₄¹, and HPO₄², and PO₄⁻³) as counter anions matched to organosilane and/or promoter cations (e.g. Na). In another embodiment the a mixture of phosphoric acid, NaH₂PO₄, and tris(trimethylsilyl)phosphate can be used to modify an inorganic oxide surface. The Group 2 or Group 1 promoter cation is added to the inorganic oxide support in the amount of about 0.3 to about 4 wt-% based on the promoter element. In other embodiments the silyl reagent is used to balance the promoter cation charge, for example [Me₃SiO]₂Ca.

Methods and catalyst compositions are provided herein for conversion of alkenols (e.g., bio-alkenols) or diols (e.g., bio-diols) to dienes (e.g., bio-dienes). In methods described herein, an aqueous mixture (e.g., a mixture that contains at least one alkenol or diol and water) is contacted with a solid phase catalyst, which produces a product stream containing at least one diene with high chemical selectivity (e.g., >90%) and an amount of water equal to the input water (e.g., steam) plus the stoichiometric amount of water (e.g., steam) produced from the dehydration reaction, shown schematically below:

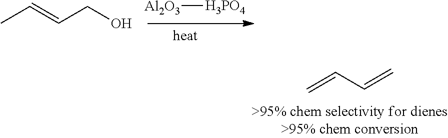

Typically, the catalytic reaction is performed at an elevated temperature (e.g., from about 200° C. to about 440° C.). Catalysts that may be used in the methods described herein include but are not limited to use of Ca⁺² as a promoter on the inorganic support surface, such as Ca⁺²-modified γ-alumina, and can also optionally include an organosilane component. Catalyst compositions for use in the methods herein include at least one acid component. In some embodiments, at least one Group 1 or Group 2 element (i.e. cation) is present at about 0.2 to about 3.5 wt-% of the modified inorganic support. In all catalysts of this invention the acid component is used from 0.1 to about 5 wt-% of the modified support, with 0.2 to 3 wt-% a preferred range.

Catalyst systems and methods of use are provided that effect a highly chemo selective dehydration of alkenols (e.g., bio-alkenols) and diols (e.g., bio-diols) in water-containing mixtures to at least one dienes with retention of chemo selectivity and catalyst activity over extended periods of use, commonly referred to as time-on-stream (TOS).

The terms “terminal alkene,” “α-olefin,” “terminal olefin,” and “1-olefin” are used interchangeably herein to refer to an alkene with a double bond between the terminal carbon (carbon at the end of a hydrocarbon chain).

The terms “internal alkene,” and “internal olefin,” are used interchangeably herein to refer to an alkene that is not terminal, i.e., the carbon-carbon double bond, is not at the end of the hydrocarbon chain. 2-Butene is one example of an “internal alkene”.

A “diene”, of which 1,3-butadiene is one example, refers to a molecule having two alkene (carbon-carbon double) bonds. In one embodiment, a “bio-diene,” e.g., “bio-1,3-butadiene” is a diene that is produced from a starting molecule (e.g., an alkenol) that is biologically produced (e.g., via microbial fermentation process). In some embodiments, a bio-olefin, bio-diene, or biobutadiene is produced from a bioalkenol that has been produced in a microbial fermentation process from sugars and waste chemical gases.

A “bio-2-olefin alcohol” or “bio-2-alken-1-ol” refers to an olefin produced from a biologically process (e.g., via microbial fermentation). The “bio-2-alkenol” can also be a product from a single dehydration step of a bio-diol. For example, bio-1,3-butanediol can be dehydrated to form bio-2-buten-1-ol, the latter is an example of a bio-2-alkenol.

A “1-alcohol” or “terminal alcohol” refers to an alcohol with a hydroxyl group attached to a terminal and primary carbon (i.e., a —CH₂OH group).

An “alkenol” refers to a molecule containing one olefin and one alcohol functional group. A “bio-alkenol” is produced using a biological process (e.g., via microbial fermentation).

“Bio-n-butanol” or “bio-1-butanol” refers to n-butanol that is produced biologically (e.g., via microbial fermentation). In some embodiments, the bio-1-butanol is produced in a microbial fermentation process from cellulosic biomass (e.g., lignocellulosic biomass) as a starting material.

Bio-alken-1-ol refers to a terminal alcohol that is produced biologically (e.g., via microbial fermentation) and contains one olefin in the molecular structure. In some embodiments, the bio-alken-1-ol is produced in a microbial fermentation process from sugars (1^{st} or 2^{nd} generation) as a starting material. In other examples, the bioalkenol can be produced from a feedstock of waster gases (e.g. carbon monoxide).

2-buten-1-ol refers to crotyl alcohol. No assignment to stereochemistry is assumed nor implied in this general description for the alkene bond. Unless directly assigned, the crotyl alcohol is assumed to be a mixture of geometric isomers (i.e., cis and trans). “Bio-2-buten-1-ol” or “bio-crotyl alcohol” refers to biologically produced bio-2-buten-1-ol (crotyl alcohol).

For α-olefins, alkenes, dienes, alcohols, diols, solvents, alkenols, bioalkenols, and all chemical reagents used in descriptions within this document, IUPAC and/or standard organic chemical nomenclature used and accepted by the American Chemical Society (ACS) takes priority and is used in a way to match common nomenclature for clarity in the disclosure.

“Biofuel,” “biolubricant,” or “bio-1,3-butadiene” refers to a fuel, a lubricant, or 1,3-butadiene, respectively, that is produced from at least one biologically-produced starting molecule or at least one starting molecule in which at least one carbon atom is derived from a biological material, for example, dextrose. Also the direct biological conversion of carbon dioxide and/or carbon monoxide is considered also defined as a viable route to bioalkenols or bio-diols. In one embodiment, a biofuel, biolubricant, or bio-1,3-butadiene may be produced from a bioalkenol that is produced by a biological process as described herein. Each of the bio-products will have a unique carbon-13/carbon-12 ratio that is dependent upon the feedstock and chemical process(s) described herein.

A “bio-” prefix is used to indicate a molecule or class of molecules, for example bio-alkenols, can be a direct product of an organism or they can be prepared through a chemical process that uses a chemical feedstock obtained from a biological process. For example, crotyl alcohol obtained by fermentation is chemically dehydrated to form a bio-1,3-butadiene.

“Regio selectivity” refers to selective formation of one olefin product over another. For example, formation of 90 mol-% terminal (1-olefin) and 10 mol-% internal (e.g., 2-olefin) would be described as 90% regio selectivity for the terminal olefin product.

“Chemo selectivity” or “chemical selectivity” refers to a specific chemical product being formed selectively over other potential products if not otherwise defined. For example, if crotyl alcohol is dehydrated to 90 mol-% 1,3-butadiene and 10 mol-% of other products, then this reaction has a 90% chemical selectivity for 1,3-butadiene.

“Chemical Yield” and “Yield” are calculated by multiplying the conversion (percent) by the chemical selectivity (percent). For example, if reaction A that has a 90% conversion with a 90% chemical selectivity for product X, then product X has a 81% chemical yield in reaction A.

“Incipient Wetness Impregnation” (IWP) is a technique that is used to modify a support material by treating the solid material with a solution containing at least one desired modifier (e.g., calcium acetate), sufficient to wet the entire solid surface and possibly fill the pore-volume. The solvent(s) used in the IWP process can be removed by application of heat, or reduced pressure, or both leaving behind the modifiers dissolved in the IWP solution.

“Weight Hourly Space Velocity” (WHSV) is defined as the weight of chemical feedstock entering the reactor per hour divided by the weight of catalyst used. For example, if 10.0 kg/hr of crotyl alcohol is fed to a reactor containing 2.0 Kg of catalyst, the WHSV is 5 hr⁻¹. The substrate feed rate, in units of WHSV, does not include any co-feeds, such as water. For the above example then, a feed of 10 Kg of crotyl alcohol containing 1.5 Kg of water (i.e. so net feed of 11.5 Kg) to a 2.0 Kg sample of catalyst would still have a WHSV of 5 hr⁻¹.

“Anions” refers to a mixture of anions necessary to counter balance cations present in solution. Anions by definition can also be considered “conjugate bases.”

“Ph” herein refers to a phenyl (C₆H₅—) group.

“Me” herein refers to an methyl (CH₃—) group.

“Et” herein refers to an ethyl (CH₃CH₂—) group.

“Ac” herein refers to an acetate (CH₃CO₂—) group.

“Aryl” herein refers to a general definition of an aromatic substituent, phenyl would be considered to be an “aryl” group.

“pKa” is defined as the −log [Ka] for an acid where Ka=[H+][A−]/[HA].

“H+-Cat” is defined as an invention catalyst prepared by modifying an inorganic support with at least one acid having a pKa of 2.5 or less. Silanized-H+-Cat refers to an invention catalyst made by modifying an inorganic support surface with at least one acid and at least one organosilane. Promoter-Silanized-H+-Cat refers to an invention catalyst prepared by modifying an inorganic support with at least one promoter, at least one acid, and at least one organosilane.

One embodiment for the dehydration catalysts disclosed herein is an inorganic support modified by contact with a Mg⁺² compound. One embodiment is to deliver the calcium compound and acid solution to the inorganic support using incipient wetness impregnation. Other embodiments can deliver a calcium compound and acid by spraying the solution on to an inorganic support. The material may be carried on a belt system or done while it is mixed in a rotating drum. Vapor phase deposition is also a possible method of depositing modifiers on an inorganic support. Solvents used to prepare the solution may be water, or one or more organic solvent(s), or a mixture thereof. Nonlimiting examples of calcium-containing compounds that may be used for production of the catalysts described herein include Ca(SO₄), Ca(HSO₄)₂, Ca(HPO₄), or Ca(H₂PO₄)₂. Other calcium and Group 2 compounds, or Group 1 compounds, or Group 3 compounds, or Group 4 compounds, or rare earth compounds may be employed for this purpose as promoters when used in conjunction with at least one acid.

In addition to at least one or more acids used to modify the inorganic oxide surface, one or more promoters can be used. These invention catalyst compositions are defined as promoter-H+-Cat compositions and disclosed herein include cations from Group for Group 2 with anions selected from a list of typical inorganic and organic conjugate bases. The promoter-H+-Cat utilizes counter cations that are advantageous for the dehydration reaction, for example Ca⁺², or it can include a mixture of just Group 2 or just Group 1 metal cations or a mixture of both Group 1 and Group 2 ions. In some embodiments, the promoter-H+-Cat includes phosphate ions, sulfate ions, or transition metal oxides which all have a well-established history of use in preparing aqueous buffer solutions that contain acids and conjugate bases.

In another embodiment, promoter-H+-Cat is prepared by adding the promoter using at least one Group 1 or Group 2 metal counter cation and an anion whose conjugate acid has a pKa of 2.5 or less. For example, Mg(HSO₄)₂ is such an example where the promoter and acid are delivered as a single component. Alternatively, if the conjugate acid of the anion does not have a pKa of 2.5 or less, then at least one acid having a pKa of 2.5 or less is added in addition to the promoter-anion. The promoter metal ion concentration is delivered at about 0.3 wt-% to about 4 wt-% of the inorganic oxide support weight and is calculated based on the promoter element weight. One non-limiting example of a suitable anion to accompany a promoter is hydrogen sulfate (HSO₄¹) since it has a pKa of about 2. Having selected SO₄² as an anion to the promoter would require addition of at least one acid such as the corresponding free acid (H₂SO₄), in combination with the selected anion(s), and organosilane may be used to prepare the catalyst. In some embodiments the free-acid, for example H₃PO₄, is added as part of the IWP solution modifying the inorganic ixude surface. In some embodiments the organosilane is tris(trimethylsilyl)phosphate, (Me₃Si)₃PO₄:

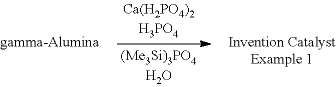

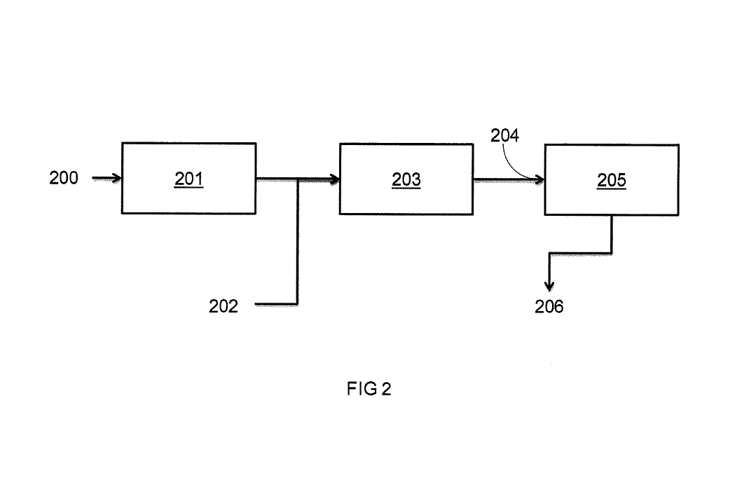

In some embodiments, a mixture of at least one acid and at least one organosilane is dissolved in one or more solvent(s) to create a pH-Cat solution, the pH-Cat solution is placed in contact with the carrier (e.g., γ-alumina), and the solvent(s) is then removed, e.g., with heat and/or reduced pressure. Those experienced in the methods of surface modification know that one technique to accomplish this is referred to as incipient wetness impregnation (IWP). IWP is typically followed by a calcination process of some time, as shown schematically in FIG. 2. The inorganic support (200) is added to vessel 201 and then addition of the IWP pH-Cat solution 202, which includes at least one acid and one organosilane (i.e. pH-Cat), with added metal promoters, is done in such a way as to provide even distribution. This can be done, for example, by contacting 202 by a spraying method. Mechanical mixing in apparatus 203 can also be used facilitate even distribution of the promoter solution. The wetted material in 203 is then heated to remove some or all of the IWP solvent. This material 204 can be further heated in a second apparatus (205) to provide calcination of the catalyst. The calcination can be carried out in vessel 203 or 205 at temperatures of about 100° C. to about 400° C., or about 200° C. to 300° C. for a time period of about 1 h to about 8 h, or about 2 h to 6 h. The removal of the solvent can be done in air or under an inert atmosphere. In one embodiment the wetting solution is removed under a reduced pressure. The catalyst removed from 204 or 205 can be sized by sifting or other methods common to solid phase catalyst processing, thus producing finished catalyst 206. The concentration of silicon in the solution is predetermined in such a way that a targeted amount of silicon is deposited on the alumina surface. In some embodiments, the silicon is deposited in the range of about 0.01 wt-% to about 2.0 wt-% or about 0.1 wt-% to 1.0 wt-% of the total catalyst {weight of atomic Si/(weight of inorganic support+weight of Ca⁺²)×100=wt-%}. Delivery of the silicon in the form of an oraganosilane and acids can also be made using other techniques such as, non-IW impregnation followed by calcination in air or under an inert atmosphere. In some embodiments, all of the modifications are carried out on the final shape or sized support material.

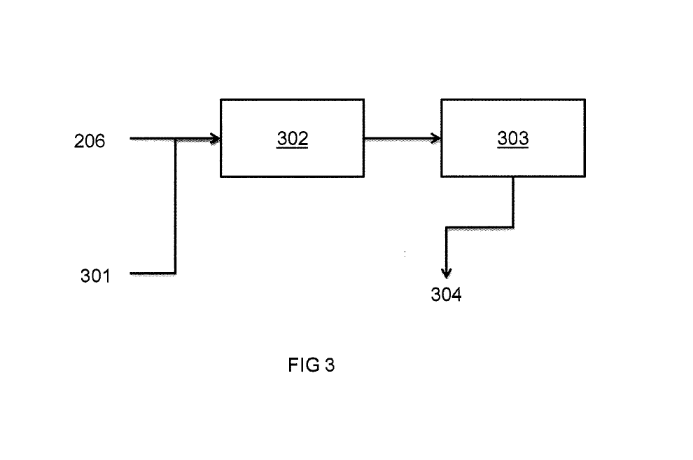

In some embodiments, the IWP solution is applied to a shaped inorganic support (e.g., γ-alumina or silica). Treatment with organosilane provides a catalyst with improved stability for the dehydration of alcohols. In some embodiments, the organosilane is delivered to the shaped or formed inorganic support using IWP, as shown in a high level and general view in FIG. 3. In this embodiment a shaped inorganic support 300 is treated with the IWP solution 301 in such a way as to provide even distribution. Since the shaped catalyst is larger compared to powder more effort is made to achieve full wetting within the porous structure. This can be accomplished by spraying the IWP solution 301 while mechanically mixing the carrier in vessel 302. The mixing process can greatly facilitate achieving an even distribution of the IWP solution. However, it is not always necessary. The wetted material in apparatus 302 is then heated to remove some or all of the IWP solution and/or can be transferred to vessel 303 for further drying under ambient or reduced pressure. The catalyst (304) can be formulated, shaped, and sized by sifting or other methods common to solid phase catalyst processing. In one embodiment, a shaped or sized support (e.g. 300) may be used in the modification process, hence providing the catalyst 304 in an advantageous physical state (e.g. shape or particle distribution).

The solution 301 used to modify the carrier by IWP can be made using water, one or more organic solvent(s), or a mixture of water and organic solvent(s) with or without additional additives. In one embodiment, the solvent is selected so that sufficient organosilane is soluble in the chosen solvent to deliver the required amount of organosilane to the solid by IWP. Typical concentrations of the organosilane reagent when used in an IWP solvent are about 0.05 wt-% to about 3 wt-%, about 0.1 wt-% to about 0.2 wt-%, about 0.15 wt-% to 1.5 wt-%, or 0.3 wt-% to 1.3 wt-% based on the element Si. Contact time for the IWP solution with modified carrier may be about 10 min to about 4 h, about 1 h to about 3 h, or about 1.5 h to about 2.5 h, thus allowing for complete dispersion and reaction, at or about ambient temperature (e.g., about 20° C. to about 30° C.). Solvent(s) removal is accomplished by the application of heat and/or reduced pressure. The typical final temperature reached is about 40° C. to 200° C., or about 300° C., or about 400° C. Removal of the solvent(s), if used, can be done in air or under an inert atmosphere (e.g., nitrogen or argon).

Typical organosilanes for use in the catalyst preparation methods disclosed herein include, but are not limited to, acetate-silanes, for example, Ph₂SiAc₂ and Ph₃SiOAc. In some embodiments, alkoxysilanes may be used. For example, organosilanes such as Ph₃SiOEt and Ph₂Si(OEt)₂ can also be used to create a silanized-dehydration catalyst. Furthermore, R₃SiX, R₂SiX₂, and RSiX₃ organosilanes can be used, where R is a mixture of aryl and alkyl radicals where X represents a leaving group that imparts solubility, or permits balancing the charge of the promoter ions, or can chemically react with water or solvent(s) to afford a second organosilane suitable for modifying an inorganic oxide surface. The R-groups can be the same or a combination of alkyl, aryl, or organic functionalized substituents. Typical leaving groups include, but are not limited to halide, alkyl carboxylate (e.g. acetate), alkoxy (e.g. methoxy), or alkyl di- (e.g., oxalate) and tri-carboxylates (e.g., citrate), and silyl-phosphates or silyl-phosphites.

In some embodiments, water-soluble organosilanes are employed in the silanization process (defined as the treatment of the support with an organosilane solution and calcining). For these embodiments, the requirement is that the organosilane be soluble in water, for example, at levels of up to about 10 wt-%. The water solubility can be achieved by the use of hydrogen bonding groups attached to the silicon atom [e.g. PhSi(OH)₃] or by incorporation of a hydrogen bonding group or a dipolar functional group to one or more of the groups attached to the silicon atom. Examples could include, but are not limited to, an alkyl amine, ether, epoxide, or other dipolar or hydrogen bonding functional groups. Solubility of the organosilane can be increased by heating of water solution to about 40° C., or about 50° C., or about 60° C., or to at least about 90° C. In some embodiments the organosilane carries a least one negative charge, for example Me₃SiO(−1) and cation is NH₄ (+).

The effect and benefit of silanization is dependent upon the carrier used, alkenol or diol feed, and the wt-% of water content added or contained in the alcohol feed. Typically, reaction conditions employing high water concentrations (e.g., >15 wt-%) in the feed may exhibit the greatest benefit from silanization of the catalyst and afford very stable dehydration catalysts for preparing dienes and maintaining high catalyst activity. Silanization of acid treated and thus modified aluminas provide a catalyst that provides enhanced stability, which is reflected in retention of selective diene (e.g., bio-diene) production as disclosed herein. The process of silanization can be applied to γ-aluminas or other inorganic supports (e.g., zinc aluminate). The silanization process can be carried before, during, or after delivery of at least one acid to the inorganic support.

Catalyst products 206 may be left as a powder, for example, in the range of about 10 to about 1000 microns, or about 50 to about 200 microns, or they can be pelletized or shaped using techniques common to those skilled in the art. In some embodiments, pellets and shaped particles may provide advantages in controlling the pressure drop across the catalyst reactor bed. This may be important where physical process requirements require long reaction tubes or where low head-pressures are desirable. Pellets may be any shape or size. In some embodiments, pellet diameters are about 1/16 in. diameter to about ¼ in., with lengths about 1 to about 10 times the diameter, or about 2 to about 4 times the diameter. One embodiment includes first shaping, or pelletizing, or crushing with size selection (e.g. sieving) of the support, followed by the addition of at least one one acid and at least one organosilane as described herein.

In methods for dehydration of a alkenol using the catalyst compositions disclosed herein, the water content in the initial alkenol (e.g., bioalkenol) or diol (e.g., bio-diol) mixture (alkenol-water or diol-water mixture) that is fed to the catalyst can be from about 0.1 wt-% to about 93 wt-%, about 1 wt-% to about 70 wt-%, about 10 wt-% to about 50 wt-%, about 15 wt-% to about 19 wt-%, or about 17 wt-%. The water in the alkenol or diol feed can be contained in the alkenol-water mixture or diol-water mixture, or added using a second and separate feed, or may be a combination of the two {e.g., total water in feed=(alkenol+water mixture)+water feed}. In some embodiments, additional water is added to the alkenol-water or diol-water mixture prior to contact with the catalyst, thereby producing an alkenol or diol feed that contains about 15 wt-% to about 90 wt-% of water, or about 45 wt-% to about 70 wt-% of water. The use of high water content in the alkenol or diol feed is especially well tolerated with catalysts prepared as disclosed herein. In some embodiments, the alkenol or diol feed in contact with the catalyst (e.g., optionally including additional water added to the alkenol-water or diol-water mixture prior to entering the reactor and/or water added separately into the reactor) includes about 1 wt-% to about 70 wt-% water for optimum production of diene (e.g., bio-diene), e.g., 1,3-butadiene (e.g., bio-1,3-butadiene). In some embodiments, the alkenol or diol feed includes >90 wt-% water. In some embodiments, the water content in the alkenol or diol feed is about 1, about 5, about 10, about 15, about 20, about 25, about 30, about 35, about 40, about 45, about 50, about 55, about 60, about 65, about 70, about 75, about 80, about 85, or about 90 wt-% or more. In some embodiments, the water content in the alkenol or diol feed is about 15 to about 20 wt-%, about 15 to about 70 wt-%, or about 50 to about 80 wt-%. The diene (e.g., bio-diene) product stream of the reaction may contain an amount of water as described above in this paragraph (e.g., 0.1 wt-%) to about 93 wt-%, or values or subranges described above that are greater than the stoichiometric amount of water that is produced in the dehydration reaction.

Catalysts disclosed herein may dehydrate wet alkenol (e.g., bio-alkenol) or diol (e.g., bio-diol) mixtures that contain large amounts of water. In some embodiments, steam may be added prior to contact with the catalyst to afford high selectivity for the diene (e.g., bio-diene) dehydration product. Furthermore, in some embodiments, a high selectivity may be maintained for extended periods of time on stream, for example, 1 to 3 months, or 6 months, or 12 months, or 18 months, or 24 months, or longer.

Catalysts disclosed herein may also dehydrate an alkenol (e.g., bio-alkenol) or diol (e.g., bio-diol) mixture that contains water at levels as low as 1 ppm to 1000 ppm where equal performance of catalyst to afford diene (e.g., bio-diene) is observed throughout the entire range of low water content and the catalyst can maintain high selectivity for extended periods of time on stream, for example, 1 to 3 months, or 6 months, or 12 months, or 18 months, or 24 months, or longer.

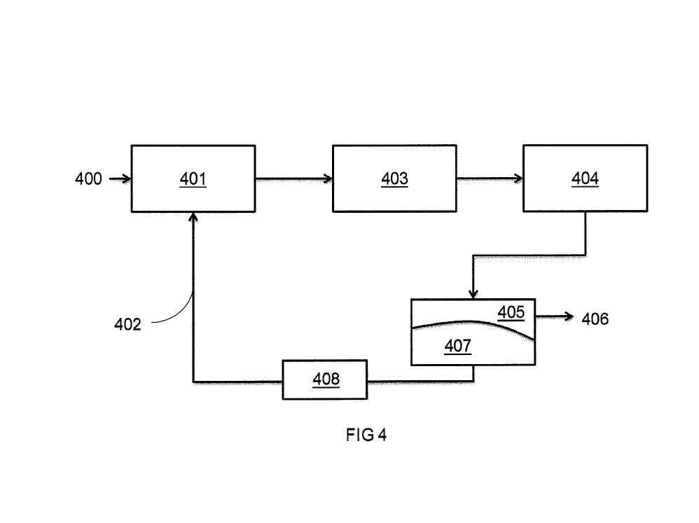

An example of a catalytic alkenol or diol dehydration process as disclosed herein is shown schematically in FIG. 4. An alkenol (e.g., bio-alkenol) or diol (e.g., bio-diol) feed 400 is introduced to the vaporization/mixing vessel (401) to which a water feed is added at some preselected amount. The output of 401 is transferred to vessel 403 which contains the dehydration catalyst (e.g., 206 or 304 prepared above) at an elevated temperature, for example, about 200° C. to about 440° C., or about 250° C. to about 420° C. Optionally, a purge gas (e.g., an inert gas such as argon and/or nitrogen) may used to control dwell time in the reactor chamber. Temperature may be modified to optimize diene (e.g., bio-diene), e.g., 1,3-butadiene (e.g., bio-1,3-butadiene) production for a particular alkenol (e.g., bio-alkenol) or diol (e.g., bio-diol) feedstock. In some embodiments, the rate of alkenol (e.g., bio-alkenol) or diol (e.g., bio-diol) feedstock passage is about 0.3 to about 10 mass units of feedstock per mass unit of catalyst/h (i.e., 0.3 to 10 WHSV), or 1.2 to 2.0 WHSV, or may be greater than 1 and less than 6 WHSV in some embodiments. Water content in the alkenol (e.g., bio-alkenol) or diol (e.g., bio-diol) feedstock in contact with the catalyst in the reactor 403 (water in the alcohol-water mixture entering the reactor, and optionally additional water added to the alcohol-water mixture and/or added separately into the reactor) is about 10 ppm (i.e., 0.001 wt-%) to about 90 wt-%. The output from apparatus 403 is fed to a cooling/condenser system 404. The output from 404 is transferred to a decanter where an aqueous solution 407 separates from the diene product 405. The isolated water product 407 (after decanting or distillation to create the isolated alkene 406) may be optionally passed through a filter material, and/or one or more other unit operations 408 to remove traces of organic acids or other unwanted byproducts that may be produced during the dehydration reaction, thereby producing a purified water stream 402. Optionally, the water stream 402 may be added to the alkenol or diol feed in 401.

In some embodiments, the alcohols (e.g., bio-alcohols) (alkenols or diols) that may be selectively dehydrated to produce dienes (e.g., bio-dienes) with the catalyst compositions and methods disclosed herein may include at least one additional organic functional group. Non-limiting examples include aromatics (e.g., with an aromatic ring that consists of carbon atoms or contains a mixture of carbon and heteroatoms), nitrile, alkene, or ketone. In some embodiments, the alcohol is a terminal alcohol. In other embodiments, the alcohol is not a terminal alcohol (e.g., 2,3-butanediol).

In some embodiments, diols are used in the dehydration methods disclosed herein. Nonlimiting examples include 1,3-butanediol, 2,3-butanediol, 1,3-pentanediol, 2,4-pentanediol, 1,5-hexandiol, 2,4-hexanediol, or 1,3-hexanediol. In one embodiment, an aqueous solution of 1,3-butanediol (e.g., containing from about 10 wt-% to about 98 wt-% of the diol) is dehydrated in a double dehydration to afford a diene as shown:

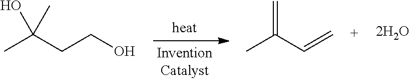

The input steam may be useful in double dehydration reactions that form a terminal olefin that is in conjugation with a second olefin bond (e.g. 1,3-butadiene). Conjugated dienes are generally more reactive and thus less stable. Dilution with solvent is commonly used to slow their decomposition/polymerization chemistry. In some embodiments an organic solvent co-feed is used.

In some embodiments of the dehydration methods disclosed herein, the alkenol or diol and water are heated to about 200° C. to 400° C. and then contacted with the dehydration catalyst. The alkenol or diol dehydration methods disclosed herein may be operated in either isothermal or adiabatic configurations. The exiting product stream may be at a temperature of about 150° C. to about 350° C. In embodiments of the dehydration methods disclosed herein, the final product stream may contain about 5 wt-% alkenol or diol or less, or less than about 0.5 wt-% alkenol or diol. Embodiments of the methods disclosed herein may produce the diene product with about 90% to about 98% or greater selectivity for the diene.

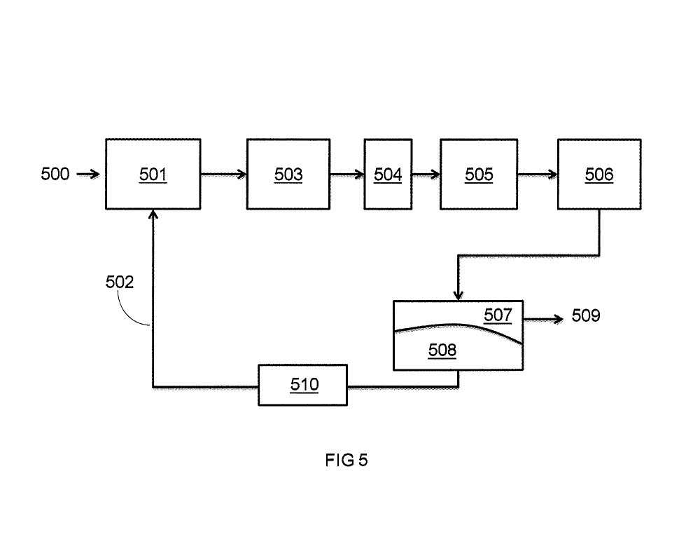

In a further embodiment, the dehydration method may include at least two reactors in sequence, with the same, similar, or better performance and yield as the single reactor system. Having the catalyst in more than one reactor can eliminate the need for external heating of the commercial reactors. Heating large commercial reactors in an isothermal manner can be cost prohibitive. In some embodiments, the product stream from the catalytic dehydration reaction (e.g., 403) is then heated again to about 200° C. to 400° C. and contacted with a second reaction vessel containing dehydration catalyst. An example of a two-reactor process is shown schematically in FIG. 5. The alkenol or diol feed 500 is added to the vaporization/mixing vessel 501 and a water feed can also added or not at some preselected amount. The output of 501 is transferred to the first catalyst vessel 503 which contains the dehydration catalyst (e.g., 206 or 304, prepared as described above). The output from 503 is reheated in 504 and fed to a second catalyst vessel 505. Vessel 503 and 505 typically do not have an external source of heat applied. The product stream from 505 is taken to a cooling/condenser system 506. The output from 506 is transferred to a decanter where an aqueous solution 508 separates from the diene product 507. The product diene 509 may be removed, and the aqueous solution 508 can be passed through one or more other unit operations 510 to remove traces of organic acids or other unwanted byproducts that can be produced during the dehydration reaction, thereby yielding a purified water stream 502. Optionally, the water stream 502 may be added to the alkenol or diol feed in 501. For those skilled in the art, this process design is commonly denoted as an “adiabatic reactor” and, in some embodiments, provides the most economical and energy efficient means of dehydrating a bioalkenol. In calculating a WHSV, the weight of alcohol feed per hour is divided by the weight of catalyst contained in both reactors. Thus in the system described above with two reactors, each separate reactor will appear to have a WHSV equaling twice that of the overall process WHSV (feed to process/total catalyst, where total catalyst is reactor 1+reactor 2; assuming, for the explanatory case described here, that each of reactor 1 and reactor 2 is charged with the same amount of catalyst).

In some embodiments of the methods disclosed herein, a device for separation of the diene product from water (e.g., an aqueous solution that contains water and other components, for example, trace organic acids and/or other byproducts) is provided. For example, water may be separated from the diene product stream by fractionation through a distillation column. Alternatively, physical separation of layers can be used when the diene product contains a sufficiently high boiling point, for example, boiling at a temperature above about 30° C. In this case, separation may optionally be achieved with a decanter (405/407 or 507/508, as shown schematically in FIG. 4 or FIG. 5, respectively). The diene product will typically be the top layer formed upon condensation and is typically siphoned from the top of the decanter vessel with the separated aqueous layer (i.e., typically bottom layer) drained (e.g., continuously drained) from the bottom layer (e.g., 407, 508. The separated aqueous layer (e.g., 407, 508) can optionally be filtered to remove impurities and the water recycled for addition to the alkenol or diol feed.

In some embodiments, the alkenol or diol/input steam is introduced to the catalyst reactor (e.g. 503) at about 1 psig to 200 psig, or about 5 psig to 150 psig, or about 15 psig to 100 psig. The exit pressure from the catalyst reactor(s) can be controlled by at least one back pressure regulator located on the exit side of the reactor. An increase in pressure can be useful although in some embodiments, this leads to a decrease in the rate of dehydration (e.g., a decrease in WHSV at a given temperature).

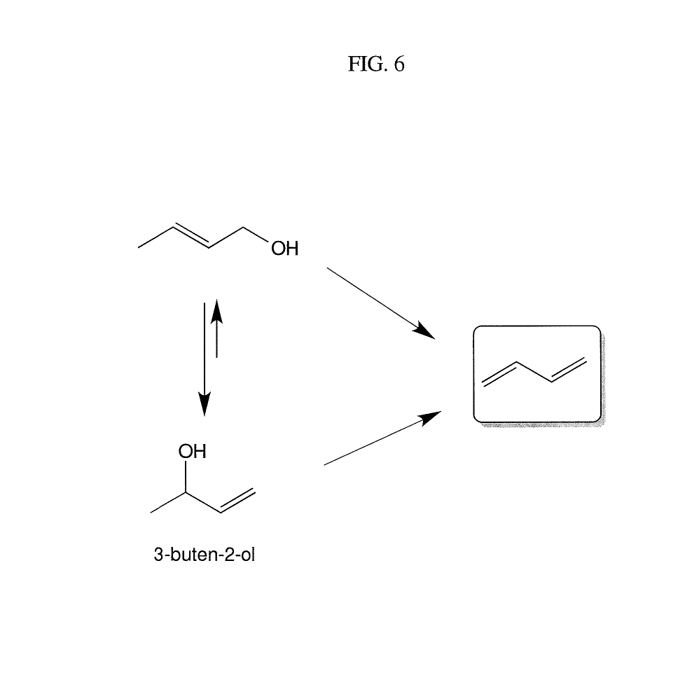

Although not wishing to be bound by theory, it is well known that crotyl alcohol undergoes a rapid isomerization reaction to form the thermodynamically favored 3-buten-1-ol [see, e.g., U.S. Pat. No. 2,373,956]. Catalytic dehydration of crotyl alcohol, isomerization may be concurrent with dehydration. In contact with the dehydration catalyst surface, isomerization is likely to occur; hence, some of the diene is likely a product from the dehydration of 3-buten-1-ol in the reactor. This reaction model is shown schematically in FIG. 6.

In some embodiments a diene product produced as described herein can be utilized to make a direct replacement diesel or jet fuel (e.g., bio fuel) or a lubricant (e.g., biolubricant) in an energy-efficient manner. The term “direct replacement” is used to indicate the new fuel prepared from the diene product can be used in commercial or military applications. For example, the new fuels could meet the requirements of ASTM D1655 (via ANNEX in ASTM D7566 for the ATJ-SPK fuels), the standard for turbine fuels. These renewable fuels are fully compatible with existing petroleum fuels used in commercial aircraft, and therefore be considered “direct replacement”. In one embodiment, the methods taught herein may be utilized for conversion of crotyl alcohol that is produced biologically, for example, in a microbial fermentation process (bio-crotyl alcohol), containing small to large amounts of water, for example, water from the fermentation broth and optionally additional added water, to 1,3-butadiene with high selectivity and chemical conversion. The 1,3-butadiene in turn is useful in preparing products that are environmentally beneficial and reduce greenhouse gases compared to petroleum equivalents by virtue of the biological production of the alcohol and an energy efficient process of making the downstream products.

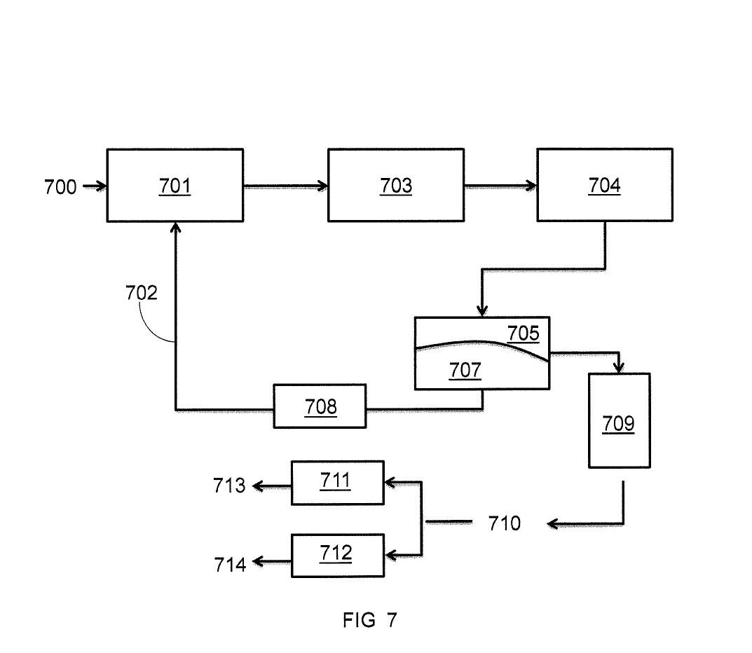

An example of a method for making 6-carbon monomers from a four-carbon alkenol or diol as the starting material is shown schematically in FIG. 7. The alkenol or diol feed 700 is added to the vaporization/mixing vessel 701 to which water feed 702 is added at some preselected amount. The output of 701 is transferred to the catalyst vessel 703 which contains the dehydration catalyst (e.g., 206 or 304, prepared as described above). The output from 703 is fed to a cooling/condenser system (704). The output from 704 is transferred to a decanter where an aqueous solution 707 separates from the diene or bio-diene product 705. The aqueous solution 707 can be passed through a filter 708 and/or one or more unit operations to remove traces of organic acids or other unwanted byproducts that can be produced during the dehydration reaction, thereby producing a purified water stream 702. Optionally, the water stream 702 may be added to the alcohol feed in 701. The diene product 705 is feed to a dryer (709) (e.g., by use of molecular sieves or similar inorganic drying agent) and subsequently undergoes a catalyzed double hydrocyanation to afford a product stream of adipoylnitrile (710). The 710 is fed to a hydrolysis reactor (711) to afford adipic acid (713) or reduced with hydrogen using a catalyst in reactor (712) to afford the products hexamethylene diamine (714). The process of converting butadiene to adipoyl nitrile, hexamethylene diamine, and adipic acid are well known and established organic synthetic transformations.

In embodiments of the dehydration methods disclosed herein in which the alkenol or diol is produced biologically (e.g., bio-1-alkenol or bio-diol), contaminants (e.g., organic contaminants) may be present in the alcohol-water mixture (e.g., from fermentation broth), including but not limited to, esters, acids, aldehydes, and/or ketones. Such contaminants can be tolerated in the dehydration reactions disclosed herein at various levels depending upon the alkenol (e.g., bio-alkenol) or diol (e.g., bio-diol) feedstock used. In some embodiments, up to about 10 ppm, or up to about 5 wt-% may be tolerated, for example, when crotyl alcohol (e.g., bio-crotyl alcohol) is starting material.

In some embodiments of the methods disclosed herein, removal of final traces of water and oxygenated organics (e.g., ethers, aldehydes, and/or aldol-type products) from the diene (e.g., bio-diene) product can be accomplished by passing the diene stream through a fixed bed of separation media, for example, activated alumina, molecular sieves, Celite, activated charcoal, size-exclusion type of media (e.g., selective gas permeation membranes), or a combination thereof. For example, the media may be contained in a vessel shown schematically in FIG. 7 as 709. In some embodiments, reactivation of the fixed-bed materials may be possible by heating under a purge of hydrocarbon or inert gas while heating to temperature of about 50° to about 200° C., or about 100° C. In one embodiment, a series of membranes and/or bed of ionic-liquids that are well known for permitting selective passage of non-polar molecules and retaining more polar organics (e.g., ethers and alcohols) can be used to dewater the diene product. A continuous and fractional distillation process can also be used to remove the higher boiling impurities from the diene product.

The following examples are intended to illustrate, but not limit, the invention.

### EXAMPLES

The engineered Clostridium strain is cultivated from a glycerol stock and inoculated into a lignocellulosic hydrolysate with defined nutrients under anaerobic conditions in a bioreactor. HPLC is used to monitor the crotyl alcohol production during fermentation. At the end of fermentation, the crotyl alcohol is separated from the fermentation broth and is used with a dehydration catalyst as disclosed herein to convert it directly to 1,3-butadiene.

### Dehydration Catalyst Preparation and Use

Catalyst 1 is prepared by modifying gamma-alumina using incipient wetness impregnation. The pH-Cat impregnation solution is prepared by dissolving 5 mL of phosphoric acid (85% in water) and 0.3 g of tris(trimethylsilyl)phosphate in 30 mL of ethanol. The solution contains a net 0.01 g of organosilane/mL. The clear solution is added to 60 g of gamma-alumina already wetted with 30 mL of ethanol and mixed for about 30 min. The impregnated carrier is then calcined at 200° C. for 6 h to obtain the final catalyst. Solvent is recovered in greater than >95% by collection by use of a cold trap.

Catalyst 2 is prepared by modifying gamma-alumina using incipient wetness impregnation and is an example of a silanized-H+-Cat invention catalyst prepared by preparing an IWP solution containing 3 mL of phosphoric acid (85% in water) and 0.6 g of tris(trimethylsilyl)phosphate in 30 mL of ethanol. The solution contains a net 0.02 g of organosilane/mL of ethanol. The clear solution is added to 60 g of gamma-alumina already wetted with 30 mL of ethanol and mixed for about 30 min. The impregnated carrier is then calcined at 250° C. for 6 h to obtain the final catalyst. Solvent was recovered in greater than >95% by collection by use of a cold trap.

Catalyst 3 is prepared by modifying gamma-alumina using incipient wetness impregnation. The IWP solution is prepared by dissolving 3 mL of phosphoric acid (85% in water) and 0.3 g of tris(trimethylsilyl)phosphate in 30 mL of ethanol. The solution contains a net 0.01 g of organosilane/mL of ethanol. The clear solution is added to 60 g of gamma-alumina already wetted with 30 mL of ethanol and mixed for about 30 min. The impregnated carrier is then calcined at 250° C. for 6 h to obtain the final catalyst. Solvent was recovered in greater than >95% by collection by use of a cold trap.

Catalyst 4 is prepared by modifying gamma-alumina extrudate using incipient wetness impregnation. The IWP solution is prepared by dissolving 3 mL of phosphoric acid (85% in water) and 0.2 g of tris(trimethylsilyl)phosphate in a mixture of 20 mL ethanol and 10 mL of water. The solution contains a net 0.01 g of organosilane/mL of ethanol. The clear solution is added to 60 g of gamma-alumina already wetted with 30 mL of ethanol and mixed for about 30 min. The impregnated carrier is then calcined at 200° C. for 4 h to obtain the final catalyst. Solvent is recovered in greater than >95% by collection by use of a cold trap.

Catalyst 5 is prepared by modifying neutral-alumina powder (Aldrich Chemical) using incipient wetness impregnation. The IWP solution is prepared by dissolving 3 mL of phosphoric acid (85% in water) and 0.2 g of tris(trimethylsilyl)phosphate in a mixture of 20 mL ethanol and 10 mL of water. The solution contains a net 0.01 g of organosilane/mL of ethanol. The clear solution is added to 60 g of gamma-alumina already wetted with 30 mL of ethanol and mixed for about 30 min. The impregnated carrier is then calcined at 200° C. for 4 h to obtain the final catalyst. Solvent is recovered in greater than >95% by collection by use of a cold trap.

Catalyst 6 is prepared by modifying gamma-alumina (Strem Chemical) using incipient wetness impregnation. The IWP solution is prepared by dissolving 3 mL of phosphoric acid (85% in water) and 0.2 g of tris(trimethylsilyl)phosphate in a mixture of 20 mL ethanol and 10 mL of water. The solution contains a net 0.01 g of organosilane/mL of ethanol. The clear solution is added to 60 g of gamma-alumina already wetted with 30 mL of ethanol and mixed for about 30 min. The impregnated carrier is then calcined at 200° C. for 4 h to obtain the final catalyst. Solvent is recovered in greater than >95% by collection by use of a cold trap.

Catalyst 7 is prepared by modifying gamma-alumina (Strem Chemical) using incipient wetness impregnation. The IWP solution is prepared by dissolving 2 mL of conc. sulfuric acid (98%) and 0.2 g of tris(trimethylsilyl)phosphate in a mixture of 20 mL ethanol and 10 mL of water. The solution contains a net 0.01 g of organosilane/mL of ethanol. The clear solution is added to 60 g of gamma-alumina already wetted with 30 mL of ethanol and mixed for about 30 min. The impregnated carrier is then calcined at 100° C. for 4 h under reduced pressure to obtain the final catalyst. Solvent is recovered in greater than >95% by collection by use of a cold trap.

Catalyst 8 is prepared by modifying gamma-alumina extrudate using incipient wetness impregnation. The IWP solution is prepared by dissolving 2 mL of conc. sulfuric acid (98%) and 0.2 g of tris(trimethylsilyl)phosphate in a mixture of 20 mL ethanol and 10 mL of water. The solution contains a net 0.01 g of organosilane/mL of ethanol. The clear solution is added to 60 g of gamma-alumina already wetted with 30 mL of ethanol and mixed for about 2 h. The impregnated carrier is then calcined at 100° C. for 4 h under reduced pressure to obtain the final catalyst. Solvent is recovered in greater than >95% by collection by use of a cold trap.

Catalyst 9 is prepared by modifying silica (Aldrich Chemical) using incipient wetness impregnation. The IWP solution is prepared by dissolving 2 mL of conc. sulfuric acid (98%) and 0.2 g of tris(trimethylsilyl)phosphate in a mixture of 20 mL ethanol and 10 mL of water. The solution contains a net 0.01 g of organosilane/mL of ethanol. The clear solution is added to 60 g of gamma-alumina already wetted with 30 mL of ethanol and mixed for about 30 min. The impregnated carrier is then calcined at 100° C. for 6 h under reduced pressure to obtain the final catalyst. Solvent is recovered in greater than >95% by collection by use of a cold trap.

Catalyst 10 is prepared by modifying gamma-alumina using incipient wetness impregnation. The IWP solution is prepared by dissolving 3 mL of phosphoric acid (85% in water) and 0.6 g of bis(trimethylsilyl)ether in 30 mL of ethanol. The solution contains a net 0.02 g of organosilane/mL of ethanol. The clear solution is added to 60 g of gamma-alumina already wetted with 30 mL of ethanol and mixed for about 30 min. The impregnated carrier is then calcined at 250° C. for 6 h to obtain the final catalyst. Solvent was recovered in greater than >95% by collection by use of a cold trap.

Catalyst 11 is prepared by modifying gamma-alumina using incipient wetness impregnation. The IWP solution is prepared by dissolving 3 mL of phosphoric acid (85% in water) and 0.6 g of diphenylsilane in 30 mL of ethanol. Slow hydrogen evolution is seen at times. The solution contains a net 0.02 g of organosilane/mL of ethanol. The clear solution is added to 60 g of gamma-alumina already wetted with 30 mL of ethanol and mixed for about 30 min. The impregnated carrier is then calcined at 250° C. for 6 h to obtain the final catalyst. Solvent was recovered in greater than >95% by collection by use of a cold trap.

Catalyst 12 is prepared by modifying gamma-alumina extrudate using incipient wetness impregnation. The IWP solution is prepared by dissolving 3 mL of phosphoric acid (85% in water) and 0.6 g of diphenylsilane in 30 mL of ethanol. Slow hydrogen evolution is seen at times. The solution contains a net 0.02 g of organosilane/mL of ethanol. The clear solution is added to 60 g of gamma-alumina already wetted with 30 mL of ethanol and mixed for about 30 min. The impregnated carrier is then calcined at 250° C. for 6 h to obtain the final catalyst. Solvent was recovered in greater than >95% by collection by use of a cold trap.

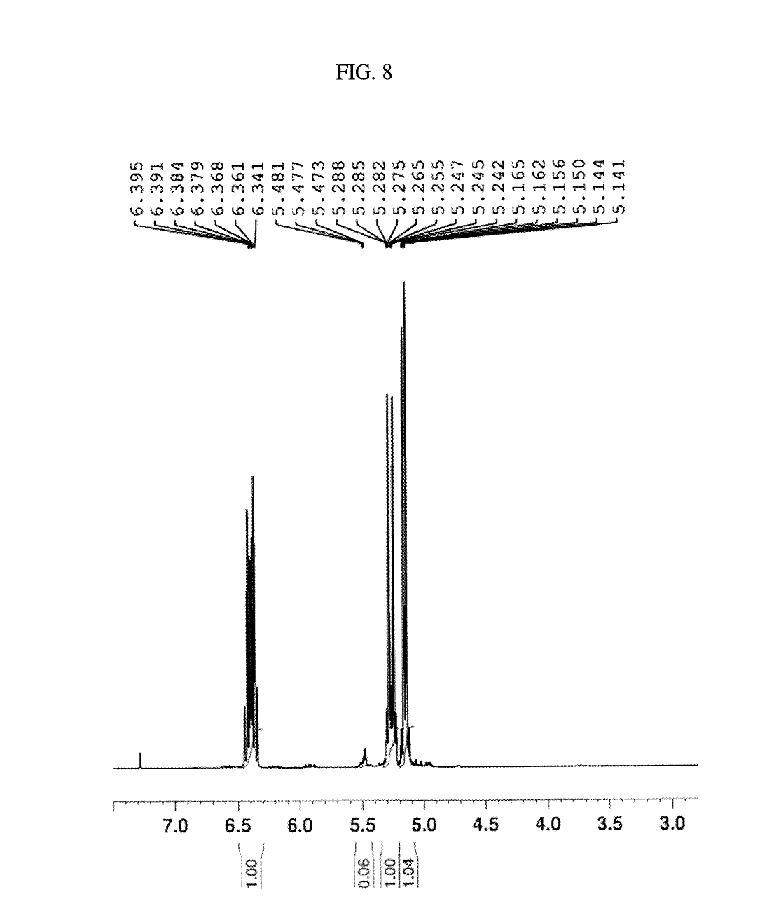

50 mL SS reactor vessel is charged with 42 g of Catalyst 2 and then each end is capped with a plug of Pyrex glass wool. The reaction vessel is heated to 250° C. using electric heating and purged under a stream of nitrogen gas during the heat up stage. Once stabilized at 250° C. for 2 h an input feed of crotyl alcohol (98%) containing 10% water is introduced at a flow rate of 0.7 g/min (based on crotyl alcohol, WHSV of 1.0). After one hour, the product stream is collected in a cold trap and then analyzed for conversion and selectivity to butadiene by GC and NMR spectroscopy. The proton NMR spectrum of the trapped 1,3-butadiene is displayed in FIG. 8.

50 mL SS reactor vessel is charged with 42 g of Catalyst 2 and then each end is capped with a plug of Pyrex glass wool. The reaction vessel is heated to 250° C. using electric heating and purged under a stream of nitrogen gas during the heat up stage. Once stabilized at 250° C. for 2 h an input feed of crotyl alcohol (98%) containing less than 0.3% water is introduced at a flow rate of 0.7 g/min (based on crotyl alcohol, WHSV of 1.0). After one hour, the product stream is collected in a cold trap analyzed and for conversion and selectivity to butadiene using GC and NMR spectroscopy.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention. And the disclosure of all articles, patents, and patent publications cited herein shall be considered to be incorporated by reference.

While the invention has been described, disclosed, illustrated and shown in various terms of certain embodiments or modifications which it has presumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

1. A silanized surface, stable, selective dehydration catalyst for dienes production, comprising (i) an inorganic support, (ii) at least one Bronsted acid of pKa less than 2.5, and (iii) at least one silicon compound.

2. A catalyst according to claim 1, further comprising at least one promoter.

3. A catalyst according to claim 1, further comprising at least one conjugate base.

4. A catalyst according to claim 1, wherein said inorganic support is gamma-alumina extrudate,

5. A catalyst according to claim 2, wherein said promoter is selected from the group consisting of Group 1 metals, Group 2 metals and combinations thereof.

6. A catalyst according to claim 3, wherein said conjugate base is selected from the group consisting of H₂PO₄⁻¹, HPO₄⁻², and combinations thereof.

7. A catalyst according to claim 1, wherein silicon concentration is in the range between about 0.01 wt % and about 2 wt % of said catalyst.

8. A catalyst according to claim 2, wherein promoter concentration is in the range between about 0.2 wt % and about 5 wt % of said catalyst.

9. A method for producing a silanized surface, stable, selective dehydration catalyst, comprising
a. providing an inorganic support;
b. mixing said inorganic support with a reagent solution comprising a solvent, a Bronstad acid, and an organosilane compound whereby a support-solution mixture is formed;
c. removing at least a portion of said solvent from said mixture to form a solvent-depleted mixture; and
d. calcining said solvent-depleted mixture, whereby said catalyst is formed.

10. A method according to claim 9, wherein said organosilane compound is selected from the group consisting of trimethylsilayl acetate, bis(trimethylsilyl) ether, diphenyldiethoxysilane, tri(trimethylsilyl)phosphate, and diphenyldiethoxysilane.

11. A method according to claim 9, wherein said solvent is selected from the group consisting of ethanol, methanol, ethylene glycol, propanediol, propanol, iso-butanol, water and mixtures thereof.

12. A method according to claim 9, wherein said reagent solution further comprises a promoter compound.

13. A method according to claim 9, wherein the silicon content of the organosilane compound is in the range between about 0.01 wt % and about 2 wt % of said support.

14. A method according to claim 12, wherein the promoter content in said reagent solution is in the range between about 0.2 wt % and about 5 wt % of said support.

15. A method for dehydrating at least one of an alkenol and a diol to a diene, comprising
a. Providing a catalyst comprising an inorganic support, at least one Bronsted acid of pKa less than 2.5, and at least one silicon compound;
b. providing a reactant mixture comprising an alcohol selected from a group consisting of alkenols and diols; and
c. contacting said reactant mixture with said catalyst at a WHSV of at least about 0.3 hr⁻¹ to selectively form a product composition comprising at least one product diene, wherein one-pass conversion yield is greater than about 80 mol %.

16. A method according to claim 15, wherein said reactant mixture further comprises water at a concentration between about 10 wt % and about 93 wt %.

17. A method according to claim 15, wherein the selectivity of said diene production is at least about 90%.

18. A method according to claim 15, wherein said alkenol comprises crotyl alcohol.

19. A method according to claim 15, wherein said product diene comprises 1,3-butadiene.

20. A method according to claim 15, wherein said contacting is conducted at a temperature in the range between about 150° C. and about 350° C.

21. A method according to claim 15, further comprising reacting said diene with at least one additional chemical to form a diene product.

22. A commercial product comprising diene product made according to the process of claim 21.

23. A commercial product according to claim 22, selected from the group consisting of synthetic rubber, plastics, copolymers and fuels.
